(19) United States
(12) Patent Application Publication
(10) Pub. No.: US 2024/0341674 A1
(43) Pub. Date: Oct. 17, 2024

(54) DIABETIC FOOT EXAMINATION DEVICE

(71) Applicant: The Research Foundation for the State University of New York, Albany, NY (US)

(72) Inventor: Ronald Miller, Syracuse, NY (US)

(21) Appl. No.: 18/635,398

(22) Filed: Apr. 15, 2024

Related U.S. Application Data
(60) Provisional application No. 63/458,991, filed on Apr. 13, 2023.

(51) Int. Cl. A61B 5/00

(52) U.S. Cl. CPC A61B 5/445; A61B 5/0013; A61B 5/0035; A61B 5/0077; A61B 5/0082; A61B 5/702; A61B 2576/02

(57) ABSTRACT

A diabetic foot examination device that monitors the progress of diabetic ulcers in a human foot based upon using, at least, visible light illumination. The device includes a body with a diagnostic pane located on an upper structure of the body and held at an angled relation to the base. The diagnostic pane is at least partially transparent to visible light and supports at least one foot placed thereupon. There is a camera affixed to the body that, at least, detects visible light and is positioned to view a foot placed against the diagnostic pane to generate diagnostic data from the detected illumination. An illumination source is affixed to the body to project light at the diagnostic pane. There is also a computer platform in communication with the camera and illumination source to control illumination, receive diagnostic data from the camera, and then create a diabetic ulcer diagnostic data.

### CROSS-REFERENCE TO RELATED APPLICATIONS

This application claims the benefit of U.S. Provisional Patent Application No. 63/458,991, filed Apr. 13, 2024, the entirety of which is hereby incorporated herein by this reference.

### BACKGROUND OF THE INVENTION

### 1. Field of the Invention

The present invention generally relates to medical diagnostic devices. More particularly, the present invention relates to a device to detect and monitor diabetic foot ulcers.

### 2. Description of the Related Art

Comprehensive diabetic medical care involves regularly scheduled clinic visits with an experienced podiatrist for foot ulcer surveillance. Frequent visits are required as diabetic neuropathy, which leads to impaired proprioceptive and nociceptive signaling along distal nerve tracks, especially in the lower extremities, is a significant feature of diabetes progression. Therefore, an appreciable and growing minority of the national population is susceptible to the development of foot ulcers. Current estimates of the national diabetes population approach 40 million Unites States citizens alone. Nearly a quarter of these individuals will develop a foot ulcer at one point in their life, with foot ulcers representing the primary inciting event that may ultimately lead to limb amputation.

Various methods and devices for early detection of tissue injury in the lower extremities, especially the plantar surface, have been implemented over the past decades. These methods range in sophistication from deep tissue imaging and real-time temperature recording to simple visual inspection. However, the most common treatment remains the combination of regular visual inspection by the patient or their partner in combination with multi-annum visits to a foot specialist trained in the management of diabetic patients. Consistency with this simple regimen has proven to be the most cost-effective approach as it does not require a great degree of technological sophistication whilst achieving adequate efficacy for per foot ulcer prevention.

Though ulcers are frequently seen daily in busy podiatric practices, most patients will present with the plantar surface completely intact and without any obvious lesions even though pre-lesions are present. Further, the patient may present with calluses or tissue changes that occur gradually over time as dictated by friction and pressure. These subtle tissue changes may easily be missed by practitioners.

There are sophisticated pressure sensor systems regularly used by podiatry clinics to diagnose issues with the plantar surface. These devices normally perform diagnostics by monitoring weight redistribution across the plantar surface during ambulation. However, minor changes which can be clinically meaningful in plantar surface structure/composition or other baseline physiological signals can be missed in these systems. It is thus to an improved diabetic foot ulcer diagnostic device that the present invention is primarily directed.

### BRIEF SUMMARY OF THE INVENTION

Briefly described, the present system and device is a diabetic foot examining device designed to fit within the paradigm of already established medical practice which could be more readily produced at scale and easily implemented into the current workflow of a footcare specialist, or deployed with a patient in a home-healthcare setting. The present technology is in the form of a frontend image capture system which acts as a structure for easily acquiring images, in addition to containing software to locally method video and image recordings to extract relevant physiological data in the assessment of acute and chronic plantar surface wounds. The build design, namely, an angled transparent diagnostic pane with sufficient surface area and imaging distance to simultaneously capture both left and right plantar surfaces using a simple off the shelf camera at a distance.

In one embodiment, the invention is a diabetic foot examination device, having a body with a base and an upper structure, and the base including a generally horizontal support configured to rest on a generally planar surface. There is a diagnostic pane located on the upper structure of the body, the diagnostic pane held at an angled relation to the base, the diagnostic pane at least partially transparent to visible light and configured to support at least one foot placed thereupon. There is also at least one camera affixed to the body and detecting illumination in the electromagnetic spectrum, with the camera positioned to view a foot placed against the diagnostic pane and generating diagnostic data from the detected illumination.

The device also includes at least one illumination source affixed to the body, that provides, at least, illumination in the electromagnetic spectrum directed at the diagnostic pane. A computer platform, which can be located in the or next to the body, is in communication with the camera and illumination source and is configured to control the illumination source to selectively direct illumination at the diagnostic pane when at least one foot is placed thereagainst, receive diagnostic data from the camera, and detect a presence of diabetic ulcers in the at least one foot based upon the received diagnostic data to create a diabetic ulcer diagnostic data.

The device can be embodied with at least one sensor connected to the computer platform, the at least one sensor detecting at least one foot being placed on the diagnostic pane. The device can also further include a wireless transmission module connected to the computer platform that selectively transmits the diabetic ulcer diagnostic data to other computer devices. Alternatively, the computer platform can be in wired communication with at least one other computer device and selectively transmit diabetic ulcer diagnostic data across the wired communication.

In one embodiment, the body of the device further includes a power source conductively connected to and powering the computer platform, camera and at least one illumination source. Further, the camera can detect visible light, or non-visible light such as UV or infrared. The diagnostic data can include multi-spectral imaging of at least one foot placed against the diagnostic pane.

Additionally, the device can include a second diagnostic device affixed to the body and selectively obtaining secondary diagnostic data from a foot placed on the diagnostic pane. The second diagnostic device can create secondary diagnostic data from one of optical coherence tomography, ultrasound scanning, Doppler scanning, autofluorescence, or laser speckle flowmetry.

In one embodiment, the invention includes a method of diagnosing diabetic ulcers in a foot utilizing the device. The method starts with placing at least one foot against a diagnostic pane of a diabetic foot examination device, illuminating the at least one foot from the illumination source, generating diagnostic data from the camera, receiving the diagnostic data at the computer platform, detecting a presence of diabetic ulcers in the at least one foot based upon the received diagnostic data, and creating, at the computer platform, a diabetic ulcer diagnostic data. The method can further include detecting at least one foot being placed on the diagnostic pane from at least one sensor connected to the computer platform.

Additionally, the method can further include selectively transmitting the diabetic ulcer diagnostic data to other computer devices, either wirelessly or in wired transmission. If embodied with a power source, the method can further include powering the computer platform, camera and at least one illumination source from a power source conductively connected thereto. The power source can be integrated into the device or be located externally thereto.

The method can include selectively obtaining secondary diagnostic data from a second diagnostic device affixed to the body, if present, and the secondary diagnostic data is for a foot placed on the diagnostic pane. The second diagnostic device can detect one or a combination of: visible light, ultraviolet light, and infra-red light.

The device can further include additional equipment to monitor physiological signals which can be captured for diagnostic purposes. These include, but are not limited to, photoplethysmography technique which extracts small signal pulsations to infer vascular flow in a tissue bed, and two-dimensional tissue oxygen saturation (SpO₂) using the modified Beer-Lambert law.

The present invention therefore is advantageous in that it can reliably diagnose diabetic foot ulcers based on, at least, precise visual image data, either in a clinic or at-home setting. The present invention also has industrial applicability in that it provides a manufacturable medical device providing useful diagnostic information. Other objects, advantages, and features of the present invention will be apparent to one of skill in the art after review of the present application.

### BRIEF DESCRIPTION OF THE DRAWINGS

FIG. 1 is a perspective view of one embodiment of the diabetic foot examination device with one foot in situ on the diagnostic pane.

FIG. 2 is a bottom view of the device embodiment of FIG. 1, with two feet placed against the diagnostic pane.

FIG. 3A is perspective view of one embodiment of the diabetic foot examination device and its components.

FIG. 3B is a side view of the device embodiment of FIG. 3A, with exemplary device dimensions noted.

FIG. 4 is a flowchart of one embodiment of a diagnostic method performed by the diabetic foot examination device.

FIG. 5A is diagram of a simple source illumination sequence of feet placed on the diagnostic pane.

FIG. 5B is a diagram of a multi-spectral illumination sequence of feet placed on the diagnostic pane.

FIG. 6 is a graph of the illumination states with corresponding diagnostic states for the scans made by the device.

FIG. 7 is a series of diagnostic scans of feet place on the diagnostic pane over a series of timeframes, the scans illustrating estimated SpO₂ during vascular occlusion.

FIG. 8 is a graph illustrating luminal relative amplitude for hallux scans in occluded and non-occluded (baseline) states.

### DETAILED DESCRIPTION OF THE INVENTION

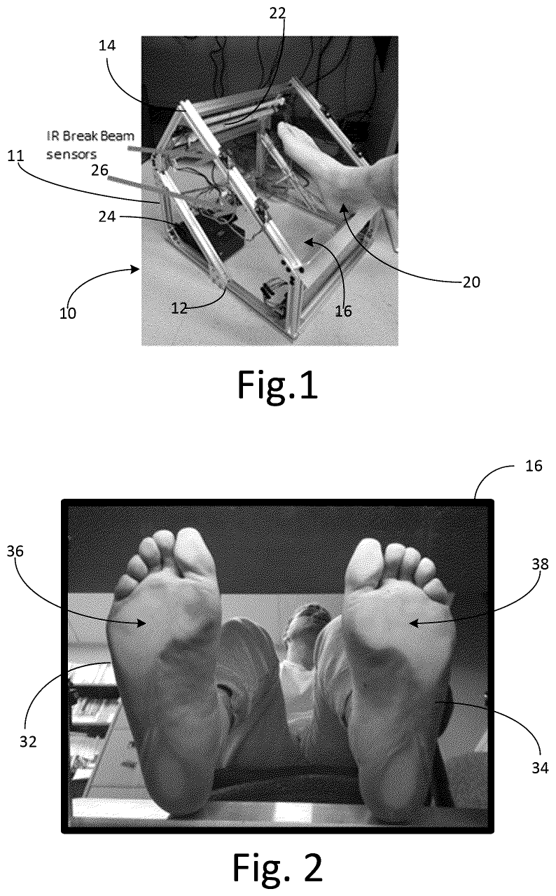

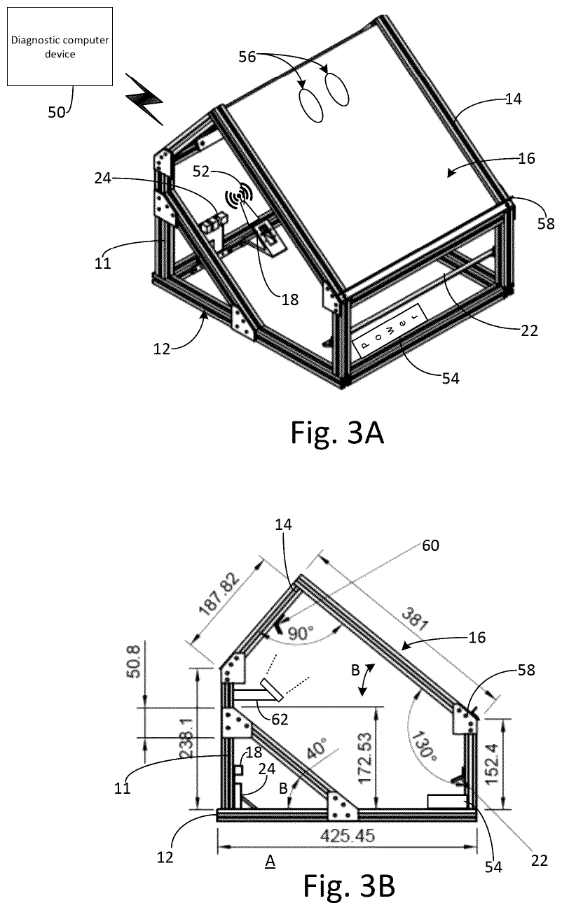

With reference to the figures in which like numerals represent like elements throughout the several views, FIG. 1 is a perspective view of one embodiment of the diabetic foot examination device 10 with one foot 20 in situ on the diagnostic pane. The device 10 has a body 11 with a base 12 and an upper structure 14, and the base 12 including a generally horizontal support (plane A, FIG. 3B) configured to rest on a generally planar surface. There is a diagnostic pane 16 located on the upper structure 14 of the body 11, the diagnostic pane 16 held at an angled relation (angle B, FIG. 3B) to the base 12, and the diagnostic pane 16 is at least partially transparent to visible light, as shown in FIGS. 1-2, and configured to support at least one foot 20 placed thereupon. There is also at least one camera 18 affixed to the body 11 and detecting illumination in the electromagnetic spectrum, with the camera 18 positioned to view a foot 20 placed against the diagnostic pane 16 and generating diagnostic data from the detected illumination.

The device 10 also includes at least one illumination source 22 affixed to the body 11, such as an LED bar or other lighting source, that provides, at least, illumination in the electromagnetic spectrum directed at the diagnostic pane 16. There is a computer platform 24, which can be located in the or next to the body 11, that is in communication with the camera 18 and illumination source 22 and is configured to control the illumination source 22 to selectively direct illumination at the diagnostic pane 16 when at least one foot 20 is placed thereagainst. The computer platform 24 then receives diagnostic data from the camera 18 and detects a presence of diabetic ulcers in the at least one foot 20 based upon the received diagnostic data to create a diabetic ulcer diagnostic data.

The device 10 can be embodied with at least one sensor connected to the computer platform, the at least one sensor 26 detecting at least one foot being placed on the diagnostic pane 16. The device 10 can also further include a wireless transmission module 52 connected to the computer platform 24 that selectively transmits the diabetic ulcer diagnostic data to other computer devices, such as diagnostic computer device 50. Alternatively, the computer platform 24 can be in wired communication with at least one other computer device, such as diagnostic computer device 50, and selectively transmit diabetic ulcer diagnostic data across the wired communication.

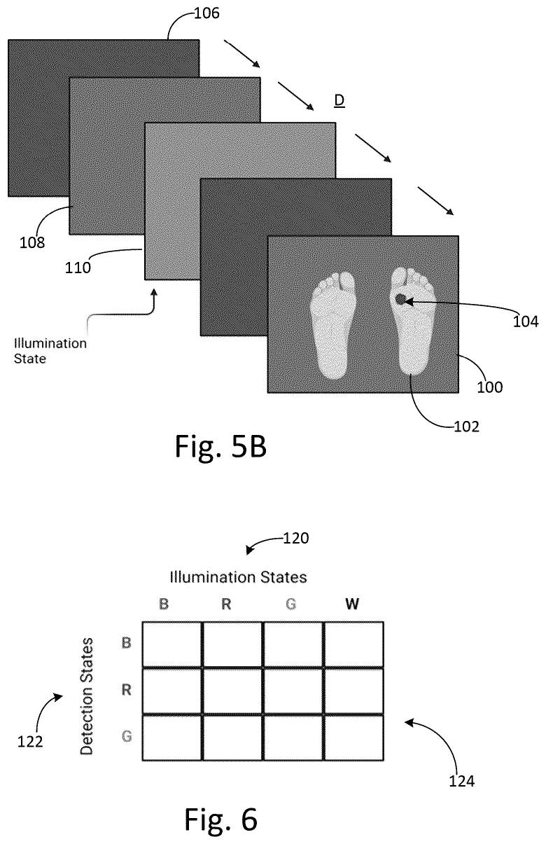

In one embodiment, the body 11 of the device 10 further includes a power source 54 conductively connected to and powering the computer platform 24, camera 28, and at least one illumination source 22. Further, the camera 18 can detect visible light, or non-visible light such as UV or infrared. The diagnostic data can include multi-spectral imaging of at least one foot 20 placed against the diagnostic pane 16, as more specifically shown in FIG. 5B.

Additionally, the device 10 can include a second diagnostic device 62 affixed to the body 11 and selectively obtaining secondary diagnostic data from a foot 20 placed on the diagnostic pane 16. The second diagnostic device 62 can create secondary diagnostic data from one of optical coherence tomography, ultrasound scanning, Doppler scanning, autofluorescence, or laser speckle flowmetry. The device 10 can therefore detect one or more of frank ulcers, regions of skin irritation/inflammation, dryness, changes in plantar surface integrity (using patient history), heart rate (pulse waveform), blood pressure (using PTT and HR current signal), or local tissue oxygenation (via multi-spectral imaging) (FIG. 5B). O₂ levels could be compared to TcPO₂ measurements.

FIG. 2 is a bottom view of the device 10 embodiment of FIG. 1, with two feet 32, 34 placed against the diagnostic pane 16. The plantar surfaces 36, 38 of foor 32 and 24 respectively, are flattened on the diagnostic pane 16 and illuminated with at least visible light to create the diagnostic data with camera 18.

FIG. 3A is perspective view of the embodiment of the diabetic foot examination device 10 of FIG. 1, but with additional components. Specifically, the device 10 includes a wireless transmitter 52 that communication diagnostic data to an external diagnostic computer device 50. The computer device 50 can be Cloud-based computing resources. Further, there is a power source 54 attached to the body, which can be a rechargeable battery, that powers the components of the device 10, such as the camera 18, light source 22, computer platform 24, and wireless transmitter 52. This allows the device 11 to be fully portable. The embodiment in FIG. 3A also includes toe marks 56, as well as a heel rest 58. These allow a patient to best align their feet (such as foot 36,38) in the diagnostic pane 16 for optimal illumination.

FIG. 3B is a side view of the device 10 embodiment of FIG. 3A, with exemplary device dimensions noted. Additional elements are present in the embodiment of FIG. 3B, sch as a second illumination device 60 that provides additional light to the diagnostic pane 16. There is also a second diagnostic device 62, in addition to camera 18, which is affixed to the body 11 and selectively obtaining secondary diagnostic data from a foot 20 placed on the diagnostic pane 16. As noted above, the second diagnostic device 62 can create secondary diagnostic data, such as from one of optical coherence tomography, ultrasound scanning, Doppler scanning, autofluorescence, or laser speckle flowmetry. Here, the imaging plate of the diagnostic pane 16 is 14 inches by 14 inches, and 0.224 inch thick acrylic and is designed to fit into 2020 aluminum slot so that the plate can be interchanged if it needs to be cleaned or replaced.

The aluminum frame for the body 11 can be made with machined aluminum brackets to fix the shown angles. The imaging plane, which can be angled between 30-60 degrees above the horizontal plane (angle B), acts as a footrest which the patient places their feet 26,38 on to be imaged by the camera 18 which is positioned at the posterior position. The distances are such that the camera, using a custom lens, captures the full extent of the image plate. IR sensors 26 flank the edge of the diagnostic pane 16 to detect the presence of a foot 20 to be imaged.

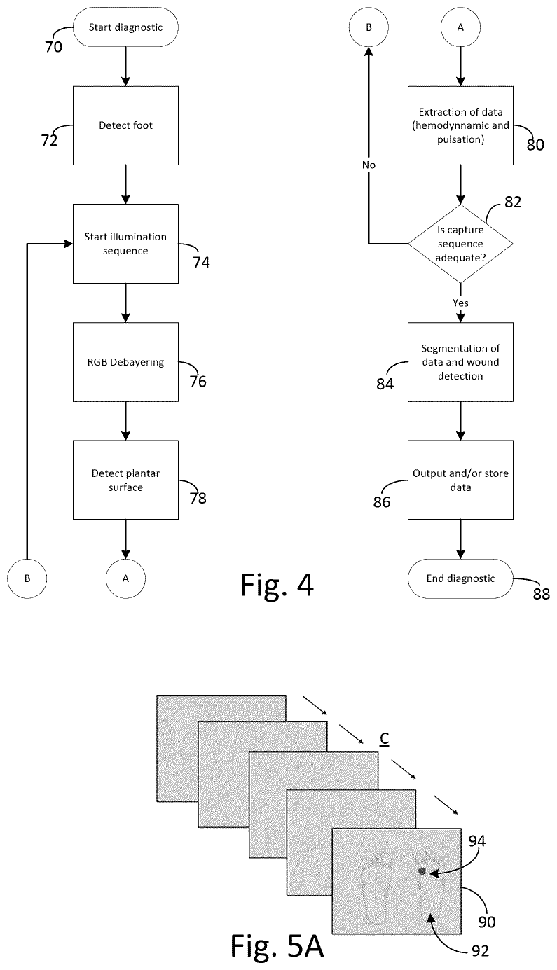

FIG. 4 is a flowchart of one embodiment of a diagnostic method performed by the diabetic foot examination device 10 to diagnose diabetic ulcers in a foot 20. The method starts with placing at least one foot 20 against a diagnostic pane 16 of a diabetic foot examination device 10, as shown at Start diagnostic 70. Then the foot 20 is detected, as shown at step 72. This detection can occur from the foot 20 breaking the IR beam of the IR sensor 26, and the illumination sequence illuminating the at least one foot 20 from the illumination source 22 occurs, as shown at step 74.

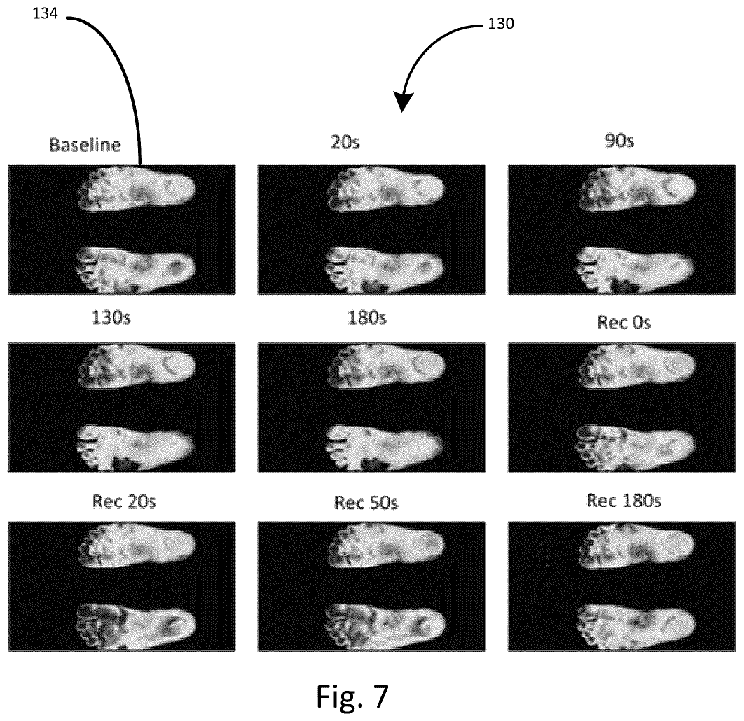

Then, in this embodiment, the method continues with RGB debayering of the illumination data from the camera 18, as shown at step 76, which can occur on the computer platform 24. The plantar surface, such as plantar surfaces 36,38 in FIG. 2 is then detected, as shown at step 78, to generate diagnostic data, such as that shown in the image sequence 130 of FIG. 7. Then the method includes generating diagnostic data from the camera 18, such as the extraction of data, shown at step 80, which can include hemodynamic or pulsation data from the image. In this embodiment, a determination is then made 82 as shown at decision 82, as to whether the capture sequence of images is adequate for diagnosis. If the received data is not adequate, then the method returns to start the illumination sequence over again at step 74.

Otherwise, if the received diagnostic data at the computer platform 24 is adequate at decision 82, the received data is segments and the method detects a presence of diabetic ulcers in the at least one foot 20 based upon the received diagnostic data, as shown at step 84. Then diabetic ulcer diagnostic data is created at the computer platform 24 and can be stored and/or output, as shown at step 86. Then the diagnostic method stops, as shown at Stop 88.

If the device 10 is so embodied such as in FIG. 3A, the method can further include selectively transmitting the diabetic ulcer diagnostic data to other computer devices, either wirelessly through wireless transmission module 52 or through a wired connection. If the power source 54 is present, then the method can further include powering the computer platform 24, camera 18, and at least one illumination source 22 from a power source 54 conductively connected thereto.

If the device 10 is so embodied such as in FIG. 3B, the method can further include selectively obtaining secondary diagnostic data from a second diagnostic device 62 affixed to the body 11, the secondary diagnostic data of a foot 20 placed on the diagnostic pane 16. The method can include the second diagnostic device 62 detecting one or a combination of: visible light, ultraviolet light, and infra-red light. The method can also include second diagnostic device 62 creating secondary diagnostic data, such as from one of optical coherence tomography, ultrasound scanning, Doppler scanning, autofluorescence, or laser speckle flowmetry.

FIG. 5A is diagram of a simple source illumination sequence C of feet 92 placed on the diagnostic pane 16. The device 10 captures images, such as image 90, of the patient's plantar surface in a streamlined and standardized manner such that the previous record of images can be analyzed to detect changes in the integrity of the plantar surface tissue, such as detection lesion 94. The foot placement angle of 50 degrees (angle B in FIG. 3B) on the diagnostic pan 16 was used for both ergonomic considerations and to limit load bearing on the imaging plate of the diagnostic pane 16.

As shown in FIG. 5A, an imaging sequence is initiated by the placement of a patients foot onto the foot plate. The foot placement is detected by two IR break beam sensors mounted on the rails of the device. A light flash is used to signal the user that the imaging sequence has begun. In one embodiment, once the imaging sequence (illumination sequence) is complete, a light signal is used to indicate that data acquisition and upload is complete, such as at decision 82 in FIG. 4.

FIG. 5B is a diagram of a multi-spectral illumination sequence D of feet 102 placed on the diagnostic pane 100. The device 10 is capable of multispectral imaging through the illumination of the tissue at separate wavelengths and extracting RGB values from the Bayer matrix of the camera 18. Quantification of ulcer 104 size is simplified by imaging the foot 102 at a constant focal plane of the diagnostic pane 16.

As shown, the image capture sequence starts with illumination source 22, which is multispectral (White, Red 106, Green 110, Blue 108), signals the patient to keep feet in place. First, the white light turns on to capture a base image, as in FIG. 3A. Next a sequence of red, then green, then blue, are used to illuminate the tissue surface sequentially. FIG. 6 is a graph 124 of the illumination states 120 with corresponding detection states 122 for the scans made by the device 10. Concurrently, the RGB camera 18 is recording at a predetermined framerate, such as 30-60 frames per second.

Once the capture sequence is complete, initial modeling begins to segment regions of the foot vs non-foot regions. A preliminary analysis is performed to determine if the next stage of modeling can proceed or whether a new capture sequence should occur. In one embodiment, the decision is signaled to the user via a light sequence (at decision 82). If the decision to proceed with analysis occurs, then the system completes a more sophisticated modeling algorithm where subregions of the foot (heel, midfoot, large toe, pinky toe, etc) are automatically identified using geometric assumptions of foot shape. Next, lesions across the plantar surface are detected using simple segmentation and multispectral weighting, as shown, such as by a vector combination of RGB in pixel space to differentiate neighboring healthy tissue from a lesioned area (ulcer 104). In addition to lesion identification, an estimate of SpO₂ can derived using the modified Beer lambert law.

The device 10 can therefore provide a lesion detection system which will identify and track current (or predict future) plantar surface lesions using a lesion classification system. The decision matrix include not only areal extent of the lesion segment, but also the position classification on the foot. For example, a lesion of equivalent area on the heel, a surface which supports a high load, would have a poorer predictive score than on the medial arch of the foot. Each area can be defined in a gradient manner not necessarily restricted to medically or anatomically defined regions, i.e., there may be patient specific morphology which causes a region of a patient's foot has a worse predictive score than another.

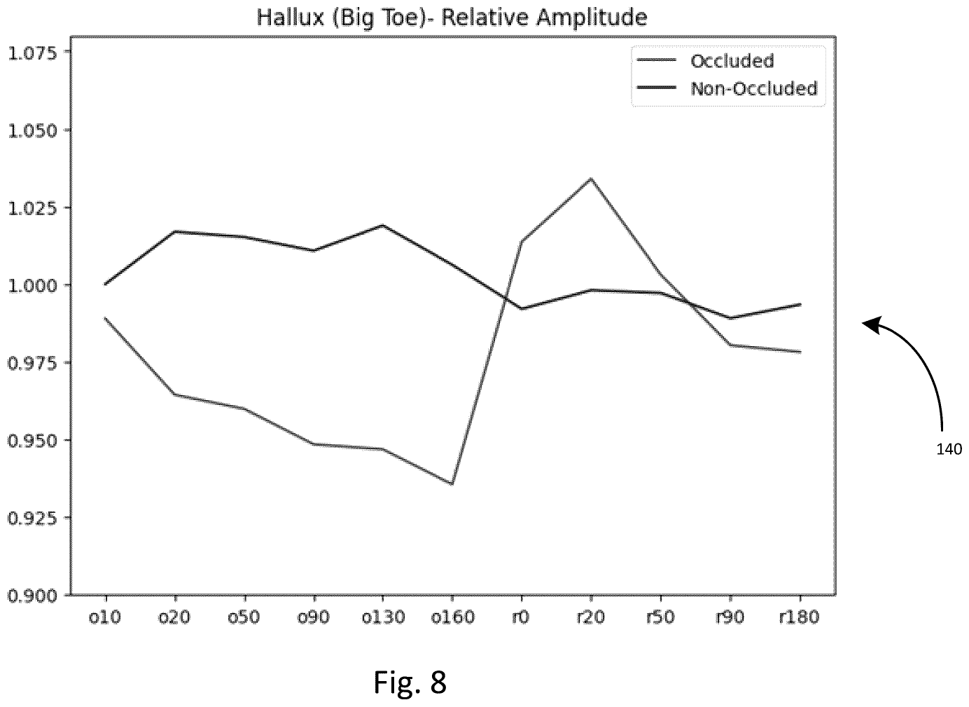

FIG. 7 is a series of image 130 of diagnostic scans of feet (32,34; FIG. 2) placed on the diagnostic pane 16 over a series of timeframes, the scans illustrating estimated SpO₂ during vascular occlusion in a global transient occlusion experiment (simulated ischemia). During this experiment, a blood pressure cuff was placed around one leg, in this case, the right leg (shown as the lower foot 132 in the images). The blood pressure cuff was sufficiently inflated (˜210 mmHg) to limit the blood flow to one foot. Throughout the occlusion, the plantar surface (36,38; FIG. 2) was imaged using the device 10. A sequence of raw images from baseline 134 was captured for 6 second periods at specified intervals before, during, and after the occlusion. We selected a resolution mode that compromised between image resolution and frame rate. Hemodynamic signals were tracked over time to derive a correlate for SpO₂. As shown by the presented images 130 and plotted data of FIG. 8, the device is capable of tracking global and focal areas of ischemia. The benefit of having stationary feet greatly enhanced the ability to implement these analyses in an automated fashion.

Local areas of tissue oxygenation (estimated by RG channel ratio reflectance) can be followed over time to quantify the recovery from the induced ischemia. The graph below shows the oxygenation correlated over time in the region of the Hallux (big toe) for both the occluded and non-occluded feet.

The vascular damage to various tissues that occurs with progression of diabetes is not evenly distributed in space or time. A multitude of factors including friction, exposure, and trauma contribute to accelerated tissue damage. Conversely, certain tissues are less affected by diabetes related vascular injury but are still subject to age related changes and other processes which are, in part, a function of anatomy (e.g., the prolonged vessel length supplying distal extremities). Therefore, comparisons of like to like tissue surfaces heightens the ability to distinguish physiologic vascular features from pathologic changes. Within the appropriate patient population (i.e. those with bilaterally intact plantar surfaces), the present invention offers a fundamental advantage: the simultaneous imaging of left and right plantar surfaces. This feature is exploited in the software processing pipeline to accentuate differences between plantar surfaces 36,38. Following low pass spatial filtering and automated labelling of plantar tissue regions, corresponding locales are compared to amplify differences in moment-to-moment tissue oxygenation and pulsation signals. This process is analogous to analog difference amplifiers which maximize differences in voltage signals as would be known to one of skill in the art.

FIG. 8 is a graph 140 illustrating luminal relative amplitude for hallux scans in occluded and non-occluded (baseline 134) state from images 130. As is shown, there is a graded decline of the RG reflectance over time during the occlusion period in the occluded foot while the non-occluded foot remains stable. The correlation between occlusion and modified RG reflectance varied across regions of the foot, but there were several areas of interest that correlated well to time within the occlusion period, making them candidates for markers of plantar surface (36,38; FIG. 2) oxygenation.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A diabetic foot examination device, comprising:
a body having a base and an upper structure, the base including a generally horizontal support configured to rest on a generally planar surface;
a diagnostic pane located on the upper structure of the body, the diagnostic pane held at an angled relation to the base, the diagnostic pane at least partially transparent to visible light and configured to support at least one foot placed thereupon;
at least one camera affixed to the body and detecting illumination in the electromagnetic spectrum, the camera positioned to view a foot placed against the diagnostic pane and generating diagnostic data from the detected illumination;
at least one illumination source affixed to the body, the illumination source providing, at least, illumination in the electromagnetic spectrum directed at the diagnostic pane; and
a computer platform in communication with the camera and illumination source, the computer platform selectively configured to:
control the illumination source to selectively direct illumination at the diagnostic pane when at least one foot is placed thereagainst;
receive diagnostic data from the camera; and
detect a presence of diabetic ulcers in the at least one foot based upon the received diagnostic data to create a diabetic ulcer diagnostic data.

2. The device of claim 1, further including at least one sensor connected to the computer platform, the at least one sensor detecting at least one foot being placed on the diagnostic pane.

3. The device of claim 1, further including a wireless transmission module connected to the computer platform, the wireless transmission module selective transmitting the diabetic ulcer diagnostic data to other computer devices.

4. The device of claim 1, wherein the computer platform is in wired communication with at least one other computer device and selectively transmits diabetic ulcer diagnostic data across the wired communication.

5. The device of claim 1, wherein the body further includes a power source conductively connected to and powering the computer platform, camera and at least one illumination source.

6. The device of claim 1, wherein the camera detects visible light.

7. The device of claim 6, wherein the diagnostic data includes multi-spectral imaging of at least one foot placed against the diagnostic pane.

8. The device of claim 1, further including a second diagnostic device affixed to the body and selectively obtaining secondary diagnostic data from a foot placed on the diagnostic pane.

9. The device of claim 8, wherein the second diagnostic device detecting one or a combination of: visible light, ultraviolet light, and infra-red light.

10. The device of claim 8, wherein the second diagnostic device creating secondary diagnostic data from one of: optical coherence tomography, ultrasound scanning, Doppler scanning, autofluorescence, or laser speckle flowmetry.

11. The device of claim 1, wherein the diagnostic pane further includes foot aligning markings.

12. A method of diagnosing diabetic ulcers in a foot, comprising:
placing at least one foot against a diagnostic pane of a diabetic foot examination device, the device comprised of:
a body having a base and an upper structure, the base including a generally horizontal support configured to rest on a generally planar surface;
a diagnostic pane located on the upper structure of the body, the diagnostic pane held at an angled relation to the base, the diagnostic pane at least partially transparent to visible light and configured to support at least one foot placed thereupon;
at least one camera affixed to the body and detecting illumination in the electromagnetic spectrum, the camera positioned to view a foot placed against the diagnostic pane and generating diagnostic data from the detected illumination;
at least one illumination source affixed to the body, the illumination source providing, at least, illumination in the electromagnetic spectrum directed at the diagnostic pane; and
a computer platform in communication with the camera and illumination source;
illuminating the at least one foot from the illumination source;
generating diagnostic data from the camera;
receiving the diagnostic data at the computer platform;
detecting a presence of diabetic ulcers in the at least one foot based upon the received diagnostic data; and
creating, at the computer platform, a diabetic ulcer diagnostic data.

13. The method of claim 12, further including detecting at least one foot being placed on the diagnostic pane from at least one sensor connected to the computer platform.

14. The method of claim 12, further including selectively transmitting the diabetic ulcer diagnostic data to other computer devices.

15. The method of claim 14, further comprising wirelessly transmitting diabetic ulcer diagnostic data.

16. The method of claim 12, further including powering the computer platform, camera and at least one illumination source from a power source conductively connected thereto.

17. The method of claim 12, further including selectively obtaining secondary diagnostic data from a second diagnostic device affixed to the body, the secondary diagnostic data of a foot placed on the diagnostic pane.

18. The method of claim 17, wherein the second diagnostic device detecting one or a combination of: visible light, ultraviolet light, and infra-red light.

19. The method of claim 17, wherein the second diagnostic device creating secondary diagnostic data from one of: optical coherence tomography, ultrasound scanning, Doppler scanning, autofluorescence, or laser speckle flowmetry.

20. A diabetic foot examination device, comprising:
a body means for supporting a base and an upper structure, the base including a generally horizontal support configured to rest on a generally planar surface;
a foot-supporting means for supporting a foot held thereagainst, the foot-supporting means located on the upper structure of the body, the foot supporting means held at an angled relation to the base, the foot-supporting means at least partially transparent to visible light;
at least one camera means for detecting illumination in the electromagnetic spectrum, the camera means affixed to the body and positioned to view a foot placed against the foot-supporting means and generating diagnostic data from the detected illumination;
at least one illumination means, providing illumination in the electromagnetic spectrum directed at the foot-supporting means, the illumination means affixed to the body; and
a diagnostic means for creating diagnostic data by:
controlling the illumination means to selectively direct illumination at the foot-supporting means when at least one foot is placed thereagainst;
receiving diagnostic data from the camera means; and
detecting a presence of diabetic ulcers in the at least one foot based upon the received diagnostic data to create a diabetic ulcer diagnostic data.
